(19) United States
(12) Patent Application Publication
(10) Pub. No.: US 2018/0077934 A1
(43) Pub. Date: Mar. 22, 2018

(54) MOLLUSCICIDE AND BAIT COMPOSITION COMPRISING A MOLLUSCICIDE

(71) Applicant: BASF SE, Ludwigshafen am Rhein (DE)

(72) Inventors: Kenichi Kojima, Raleigh, NC (US); Renee Westich, Apex, NC (US); Huazhang Huang, Durham, NC (US)

(21) Appl. No.: 15/562,747

(22) PCT Filed: Apr. 22, 2016
(86) PCT No.: PCT/EP2016/059040

§ 371 Date: Sep. 28, 2017

Related U.S. Application Data
(60) Provisional application No. 62/150,956, filed on Apr. 22, 2015.

(30) Foreign Application Priority Data
May 11, 2015 (EP) 15167190.6

(51) Int. Cl. A01N 43/78; C07D 417/14; C07D 417/04; C07D 401/04; A01N 43/56

(52) U.S. Cl. CPC A01N 43/78; C07D 417/14; A01N 43/56; C07D 401/04; C07D 417/04

(57) ABSTRACT

The use of certain pesticides for protecting plants, the plant propagation material thereof, or soil or water, in which the plants are growing, against the attack or infestation by gastropods is described. Furthermore, a method for controlling gastropods is disclosed, the method comprising contacting the gastropods or their food supply, habitat or breeding ground, or plants or plant propagation material thereof, or soil or water, where the gastropods are present or may occur, with a molluscicidally effective amount of a pesticide. A bait composition is also provided, comprising a pesticide, a mollusc attractant, and optionally at least one further additive.

In a first aspect, the present invention relates to the use of a pesticide, which is
(a) a compound of formula I

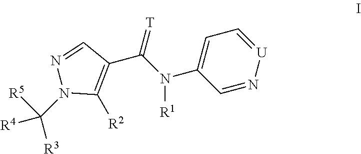

wherein
U is N or CH;
T is O or S;
R¹ is H, C₁-C₂-alkyl, or C₁-C₂-alkoxy-C₁-C₂-alkyl;
R² is CH₃, or halomethyl;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
R³ and R⁴ may together form a 3- to 6-membered carbo- or heterocycle, which may contain 1 or 2 heteroatoms selected from N—R^{c}, O, and S, wherein S may be oxidized, which carbo- or heterocycle may be unsubstituted, or partially or fully substituted by R^{a};
   wherein
   R^{c} is hydrogen, C₁-C₂-alkyl, C₁-C₂-haloalkyl, C₁-C₂-alkylcarbonyl, or C₁-C₂-alkoxy-carbonyl;
or a stereoisomer, salt, tautomer or N-oxide thereof; or
(b) a compound selected from (b1) 2-(1,3-Dioxan-2-yl)-6-[2-(3-pyridinyl)-5-thiazolyl]-pyridine; (b2) 2-[6-[2-(5-Fluoro-3-pyridinyl)-5-thiazolyl]-2-pyridinyl]-pyrimidine; (b3) 2-[6-[2-(3-Pyridinyl)-5-thiazolyl]-2-pyridinyl]-pyrimidine; (b4) N-Methylsulfonyl-6-[2-(3-pyridyl)thiazol-5-yl]pyridine-2-carboxamide; (b5) 2-(3-pyridyl)-5-[6-(2-pyridyl)-2-pyridyl]thiazole; (b6) N-Ethyl-N-[4-methyl-2-(3-pyridyl)thiazol-5-yl]-3-methylsulfanyl-propanamide; (b7) N-Methyl-N-[4-methyl-2-(3-pyridyl)thiazol-5-yl]-3-methylsulfanyl-propanamide; (b8) N,2-Dimethyl-N-[4-methyl-2-(3-pyridyl)thiazol-5-yl]-3-methylsulfanyl-propanamide; (b9) N-Ethyl-2-methyl-N-[4-methyl-2-(3-pyridyl)thiazol-5-yl]-3-methylsulfanyl-propanamide; (b10) N-[4-Chloro-2-(3-pyridyl)thiazol-5-yl]-N-ethyl-2-methyl-3-methylsulfanyl-propanamide; (b11) N-[4-Chloro-2-(3-pyridyl)thiazol-5-yl]-N,2-dimethyl-3-methylsulfanyl-propanamide; (b12) N-[4-Chloro-2-(3-pyridyl)thiazol-5-yl]-N-methyl-3-methylsulfanyl-propanamide; (b13) N-[4-Chloro-2-(3-pyridyl)thiazol-5-yl]-N-ethyl-3-methylsulfanyl-propanamide; and (b14) N-[4-chloro-2-(3-pyridyl)thiazol-5-yl]-N-cyclopropyl-2-methyl-3-methylsulfanyl-propanamide;
   for protecting plants, the plant propagation material thereof, or soil or water, in which the plants are growing, against the attack or infestation by gastropods.

In a further aspect, the present invention relates to the use of a pesticide, which is
(a) a compound of formula I

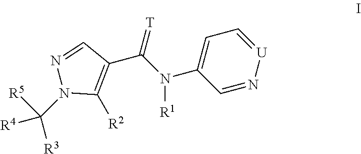

wherein
U is N or CH;
T is O or S;
R¹ is H, C₁-C₂-alkyl, or C₁-C₂-alkoxy-C₁-C₂-alkyl;
R² is CH₃;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
or a stereoisomer, salt, tautomer or N-oxide thereof; or
(b) a compound selected from (b5) 2-(3-pyridyl)-5-[6-(2-pyridyl)-2-pyridyl]thiazole; (b6) N-Ethyl-N-[4-methyl-2-(3-pyridyl)thiazol-5-yl]-3-methylsulfanyl-propanamide; (b7) N-Methyl-N-[4-methyl-2-(3-pyridyl)thiazol-5-yl]-3-methylsulfanyl-propanamide; (b8) N,2-Dimethyl-N-[4-methyl-2-(3-pyridyl)thiazol-5-yl]-3-methylsulfanyl-propanamide; (b9) N-Ethyl-2-methyl-N-[4-methyl-2-(3-pyridyl)thiazol-5-yl]-3-methylsulfanyl-propanamide; (b10) N-[4-Chloro-2-(3-pyridyl)thiazol-5-yl]-N-ethyl-2-methyl-3-methylsulfanyl-propanamide; (b11) N-[4-Chloro-2-(3-pyridyl)thiazol-5-yl]-N,2-dimethyl-3-methylsulfanyl-propanamide; (b12) N-[4-Chloro-2-(3-pyridyl)thiazol-5-yl]-N-methyl-3-methylsulfanyl-propanamide; (b13) N-[4-Chloro-2-(3-pyridyl)thiazol-5-yl]-N-ethyl-3-methylsulfanyl-propanamide; and (b14) N-[4-chloro-2-(3-pyridyl)thiazol-5-yl]-N-cyclopropyl-2-methyl-3-methylsulfanyl-propanamide;
   for protecting plants, the plant propagation material thereof, or soil or water, in which the plants are growing, against the attack or infestation by gastropods.

In a further aspect, the present invention relates to the use of a pesticide, which is
(a) a compound of formula I

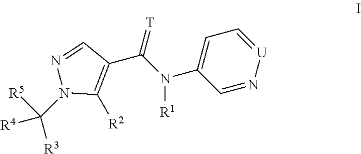

wherein
U is N or CH;
T is O or S;
R¹ is H, C₁-C₂-alkyl, or C₁-C₂-alkoxy-C₁-C₂-alkyl;
R² is CH₃;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
or a stereoisomer, salt, tautomer or N-oxide thereof; or
(b) a compound selected from (b5) 2-(3-pyridyl)-5-[6-(2-pyridyl)-2-pyridyl]thiazole; (b6) N-Ethyl-N-[4-methyl-2-(3-pyridyl)thiazol-5-yl]-3-methylsulfanyl-propanamide; (b7) N-Methyl-N-[4-methyl-2-(3-pyridyl)thiazol-5-yl]-3-methylsulfanyl-propanamide; (b8) N,2-Dimethyl-N-[4-methyl-2-(3-pyridyl)thiazol-5-yl]-3-methylsulfanyl-propanamide; (b9) N-Ethyl-2-methyl-N-[4-methyl-2-(3-pyridyl)thiazol-5-yl]-3-methylsulfanyl-propanamide; (b10) N-[4-Chloro-2-(3-pyridyl)thiazol-5-yl]-N-ethyl-2-methyl-3-methylsulfanyl-propanamide; (b11) N-[4-Chloro-2-(3-pyridyl)thiazol-5-yl]-N,2-dimethyl-3-methylsulfanyl-propanamide; (b12) N-[4-Chloro-2-(3-pyridyl)thiazol-5-yl]-N-methyl-3-methylsulfanyl-propanamide; (b13) N-[4-Chloro-2-(3-pyridyl)thiazol-5-yl]-N-ethyl-3-methylsulfanyl-propanamide; (b14) N-[4-chloro-2-(3-pyridyl)thiazol-5-yl]-N-cyclopropyl-2-methyl-3-methylsulfanyl-propanamide; (b15) N-[3-Chloro-1-(pyridine-3-yl)-1H-pyrazol-4-yl]-N-ethyl-3-((3,3,3-trifluoropropyl)thio)propanamide; (b16) N-[3-Chloro-1-(pyridine-3-yl)-1H-pyrazol-4-yl]-N-ethyl-3-((3,3,3-trifluoropropyl)sulfanyl)propanamide; (b17) N-[3-Chloro-1-(pyridine-3-yl)-1H-pyrazol-4-yl]-3-(((2,2-difluorocyclopropyl)methyl)thio)-N-ethylpropanamide; and (b18) N-[3-Chloro-1-(pyridine-3-yl)-1H-pyrazol-4-yl]-3-(((2,2-difluorocyclopropyl)methyl)sulfinyl)-N-ethylpropanamide;
   for protecting plants, the plant propagation material thereof, or soil or water, in which the plants are growing, against the attack or infestation by gastropods.

In a further aspect, the present invention relates to the use of a pesticide, which is
(a) a compound of formula I

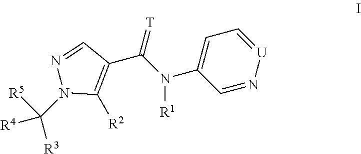

wherein
U is N or CH;
T is O or S;
R¹ is H, C₁-C₂-alkyl, or C₁-C₂-alkoxy-C₁-C₂-alkyl;
R² is CH₃;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
or a stereoisomer, salt, tautomer or N-oxide thereof; or
(b) a compound selected from (b15) N-[3-Chloro-1-(pyridine-3-yl)-1H-pyrazol-4-yl]-N-ethyl-3-((3,3,3-trifluoropropyl)thio)propanamide; (b16) N-[3-Chloro-1-(pyridine-3-yl)-1H-pyrazol-4-yl]-N-ethyl-3-((3,3,3-trifluoropropyl)sulfanyl)propanamide; (b17) N-[3-Chloro-1-(pyridine-3-yl)-1H-pyrazol-4-yl]-3-(((2,2-difluorocyclopropyl)methyl)thio)-N-ethylpropanamide; and (b18) N-[3-Chloro-1-(pyridine-3-yl)-1H-pyrazol-4-yl]-3-(((2,2-difluorocyclopropyl)methyl)sulfinyl)-N-ethylpropanamide;
   for protecting plants, the plant propagation material thereof, or soil or water, in which the plants are growing, against the attack or infestation by gastropods.

A pesticide according to option (a) or (b) as defined above is in the following referred to as pesticide (a) or (b), respectively.

In a second aspect, the present invention relates to a method for controlling gastropods comprising contacting the gastropods or their food supply, habitat or breeding ground; or plants or plant propagation material thereof, or soil or water, wherein the gastropods are present or may occur, with a molluscicidally effective amount of a pesticide (a) or (b) as defined above.

In a third aspect, the present invention relates to a composition comprising
(i) at least one pesticide (a) or (b) as defined above;
(ii) at least one mollusc attractant; and optionally
(iii) at least one further additive.

The composition is suitable as a bait composition due to the presence of the mollusc attractant.

Gastropods, such as slugs and snails, are an increasing problem in horticulture and agriculture, as they cause severe damage by feeding. Gastropods are serious pests, e.g., of cereals, rapeseed, leaf vegetables, and ornamentals. Gastropods feed both above and below the surface of the ground, on seeds, seedlings and plants, damaging shoots, roots, leaves and flowers, therefore reducing plant stand and crop yield.

Changes in the management of crops (such as minimum tillage, direct drilling, and high organic matter build up in the soil) have led to increased population densities of slugs and snails, further exacerbating this problem and resulting in more extensive and severe damage to crops.

Non-chemical methods of gastropod control include erecting physical barriers, application of predators such as beetles, or exposure to parasites such as nematodes. Whilst each of these methods has its merits, none of them provide levels of control that are as good as chemicals.

Chemical methods for controlling gastropods are mostly based on metaldehyde, ferric phosphate, methiocarb or thiodicarb as molluscicide. Of these molluscicides, methiocarb and thiocarbamat have the disadvantage of being highly toxic to mammals including, e.g., hedgehogs and pets. Furthermore, they can harm beneficial insects. Metaldehyde and ferric phosphate are less toxic to beneficial organisms, but are also less efficacious for gastropod control.

Accordingly, there is a need for further chemicals, which can be used for protecting plants from gastropods and which are suitable for controlling gastropods, and which overcome the disadvantages of the currently used molluscicides.

In connection with the current methods of gastropod control, it is another important aspect that the molluscicides are usually provided in the form of bait compositions, in particular in the form of slug bait pellets, which comprise a mollusc attractant and the molluscicide. However, pellets are often only effective for about a week and have a low stability in rainy conditions. It is therefore the general procedure to constantly monitor the crops in terms of gastropod damages and apply the pellets as needed. One problem in this connection is that damage often goes undetected when the crop is emerging and resultant poor establishment is attributed to agronomic factors. Baiting is then often applied too late and crops need to be resown.

Furthermore, baiting is often not efficient against juvenile slugs as juveniles often remain in the soil, and feed from the emerging crop below the ground.

In particular with regard to the protection of plant propagation material in particular seeds of a plant against attack or infestation by gastropods and with regard to the protection of seedlings against attack or infestation by gastropods, it would therefore be advantageous to find chemicals, which can also be applied by other means than a bait composition.

In connection with gastropod control, it is often also required to provide insect control, e.g. aphid control, as both pests often occur on the same plants. A prominent example in this regard is, e.g., rapeseed. Consequently, it would be advantageous to find pesticides which have both, insecticidal and molluscicidal activity. It is therefore another object of the present invention to provide chemicals, which are suitable for protecting plants against attack or infestation by gastropods, while at the same time controlling insects, in particular aphids.

Furthermore, it is an object of the invention to provide improved bait compositions against gastropods and uses and methods comprising the application of such bait compositions.

It has surprisingly been found that the above objects can be achieved by the uses and methods as defined herein comprising the application of a pesticide (a) or (b) as defined above, as well as by the bait composition as defined herein comprising at least one pesticide (a) or (b) as defined above, and uses and methods as defined herein comprising the application of said bait composition.

Pesticides (a), which are classified as N-(het)arylamides derived from pyrazole carboxylic acids, and their preparation are described e.g. in WO2010/034737, WO2012/084670, WO2012/143317, and U.S. 61/891,437. The compounds are known to be particularly useful as insecticides, in particular for controlling aphids, dipteral, leafhopper, thrips, and whiteflies.

Pesticides (b), which are classified as 5-membered 3-pyridiyl heterocycles, are described e.g. in WO2010/006713, WO2011/134964, WO2012/000896, WO2014/005982, WO2010/129497, WO2013/184475, WO2013/184476, and WO2013/184480. It is known in the art that 3-pyridyl thiazoles are highly effective against aphids and whiteflies.

So far, the pesticides (a) and (b) have not been described as suitable for use against gastropods. It is therefore a surprising finding of the present invention that the pesticides (a) and (b) are suitable for protecting plants, the plant propagation material thereof, or soil or water, in which the plants are growing, against the attack or infestation by gastropods. Protection is mainly achieved by repelling the gastropods, but can also be achieved by killing the gastropods, if they nevertheless feed e.g. from the plant or plant propagation material, which has been treated with the pesticide (a) or (b). The present invention therefore also relates to a method of controlling gastropods comprising contacting the gastropods or their food supply, habitat or breeding ground; or plants or plant propagation material thereof, or soil or water, wherein the gastropods are present or may occur, with a molluscicidally effective amount of a pesticide (a) or (b). It has also been found that the pesticides (a) and (b) are suitable for use in bait compositions for controlling gastropods. The gastropods will feed from the bait composition due to the presence of a mollusc attractant and will thereby ingest the harmful pesticide (a) or (b). Another possibility for controlling gastropods is the combination of (i) applying the pesticide (a) or (b) on plants or the plant propagation material thereof, and (ii) applying a bait composition comprising a molluscicide, which may either be a pesticide (a) or (b) or another molluscicide known in the art, and a mollusc attractant to the soil surrounding the plant or plant propagation material. The gastropods will then be repelled from the plants, and they will be attracted and killed by the bait composition. In addition, the pesticide (a) or (b) on the plants or the plant propagation material thereof can control aphids, so that a double effect may be achieved.

The pesticides (a) and (b) are described in further detail hereinafter.

Pesticide (a) is a compound of formula I as defined herein.

In one embodiment, pesticide (b) is a compound selected from the group consisting of compounds b1, b2, b3, b4, b5, b6, b7, b8, b9, b10, b11, b12, b13, and b14. In a preferred embodiment, pesticide (b) is a compound selected from the group consisting of compounds b5, b6, b7, b8, b9, b10, b11, b12, b13, and b14.

In another embodiment, pesticide (b) is a compound selected from the group consisting of compounds b5, b6, b7, b8, b9, b10, b11, b12, b13, b14, b15, b16, b17, and b18. In another embodiment, pesticide (b) is a compound selected from the group consisting of compounds b15, b16, b17, and b18.

In one embodiment, the term “compound(s) according to the invention” or “compound(s) of the invention” encompasses both compounds of formula I and compounds selected from the group consisting of compounds b1, b2, b3, b4, b5, b6, b7, b8, b9, b10, b11, b12, b13, and b14. Preferably, the term “compound(s) according to the invention” or “compound(s) of the invention” encompasses both compounds of formula I and compounds selected from the group consisting of compounds b5, b6, b7, b8, b9, b10, b11, b12, b13, and b14.

In another embodiment, the term “compound(s) according to the invention” or “compound(s) of the invention” encompasses both compounds of formula I and compounds selected from the group consisting of compounds b5, b6, b7, b8, b9, b10, b11, b12, b13, b14, b15, b16, b17, and b18. In another embodiment, the term “compound(s) according to the invention” or “compound(s) of the invention” encompasses both compounds of formula I and compounds selected from the group consisting of compounds b15, b16, b17, and b18.

The term “compound(s) of formula I” or “compound(s) I” comprises the compound(s) as defined herein in connection with generic formula I as well as a stereoisomer, salt, tautomer or N-oxide thereof.

The term “stereoisomers” encompasses both optical isomers, such as enantiomers or diastereomers, the latter existing due to more than one centre of chirality in the molecule, as well as geometrical isomers (cis/trans isomers).

Depending on the substitution pattern, the compounds of formula I may have one or more centres of chirality, in which case they are present as mixtures of enantiomers or diastereomers. One centre of chirality is the carbon atom carrying radicals R³, R⁴ and R⁵. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term “N-oxide” relates to a form of compounds I in which at least one nitrogen atom is present in oxidized form (as NO).

The compounds of the formula I may be amorphous or may exist in one or more different crystalline states (polymorphs) which may have different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention.

Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The following remarks made as to preferred embodiments of the variables (substituents) of the compounds of formula I, i.e. the pesticide (a), are to be understood as preferred on their own as well as preferably in combination with each other, as well as in combination with stereoisomers, tautomers, N-oxides or salts of the compounds of formula I, and in combination with the uses and methods comprising the application of the compounds of formula I as defined herein, the compositions as defined herein, and the uses and methods comprising the application of said composition.

In one embodiment of the invention, the pesticide (a) is a compound of formula I

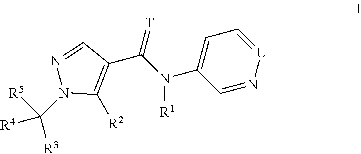

wherein
U is N or CH;
T is O or S;
R¹ is H, C₁-C₂-alkyl, or C₁-C₂-alkoxy-C₁-C₂-alkyl;
R² is CH₃, or halomethyl;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
R³ and R⁴ may together form a 3- to 6-membered carbo- or heterocycle, which may contain 1 or 2 heteroatoms selected from N—R^{c}, O, and S, wherein S may be oxidized, which carbo- or heterocycle may be unsubstituted, or partially or fully substituted by R^{a};
wherein
R^{c} is hydrogen, C₁-C₂-alkyl, C₁-C₂-haloalkyl, C₁-C₂-alkylcarbonyl, or C₁-C₂-alkoxycarbonyl; or a stereoisomer, salt, tautomer or N-oxide thereof.

In a preferred embodiment of the invention, the pesticide (a) is a compound of formula I,

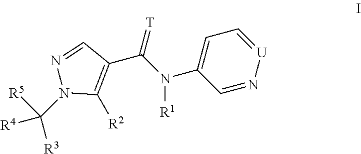

wherein
U is N or CH;
T is O or S;
R¹ is H, C₁-C₂-alkyl, or C₁-C₂-alkoxy-C₁-C₂-alkyl;
R² is CH₃;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
   wherein
   R^{a} is halogen, CN, NO₂, C₁-C₂-alkyl, C₁-C₂-haloalkyl, C₁-C₄-alkoxy, C₁-C₂-halo-alkoxy, or S(O)ₙR^{b};
   n is 0, 1, or 2; and
   R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
or a stereoisomer, salt, tautomer or N-oxide thereof.

In one embodiment, U is N.

In one embodiment, T is O.

In one embodiment, R¹ is H, CH₃, C₂H₅, or CH₂OCH₃, preferably CH₃, or C₂H₅.

In one embodiment, R² is CH₃.

In one embodiment, R³ is C₁-C₆-alkyl, C₁-C₆-haloalkyl, C₃-C₆-cycloalkyl, wherein the C-atoms may be unsubstituted, or partially or fully substituted by halogen, or CN.

In a preferred embodiment, R³ is CH₃, C₂H₅, CH(CH₃)₂, CH₂CH₂CH₃, CF₃, CHFCH₃, cyclopropyl, wherein the ring is substituted by halogen, or CN.

In a further preferred embodiment, R³ is CH₃, CH(CH₃)₂, CF₃, CHFCH₃, 1-CN-c-C₃H₄.

In one embodiment, R⁴ is C₁-C₄-alkyl, preferably CH₃.

In another embodiment R³ and R⁴ together form C₅-C₆-cycloalkyl, wherein the C-atoms may be unsubstituted, or partially or fully substituted by halogen, or CN.

In one embodiment, R⁵ is H.

In another embodiment, R⁵ is H, and R⁴ is CH₃.

In a preferred embodiment, R¹ is CH₃ or C₂H₅, R² is CH₃, R⁵ is H, and R⁴ is CH₃, and R³ is selected from CH₃, CH(CH₃)₂, CF₃, CHFCH₃, and 1-CN-c-C₃H₄.

In one embodiment, the compound of formula I is a compound of formula IA as depicted below, wherein U is N, T is O, R² is CH₃ and R⁵ is H.

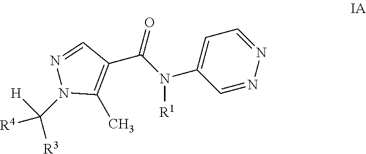

For the compounds of formula IA, it is particularly preferred that R¹ is H, CH₃, C₂H₅, or CH₂OCH₃.

Furthermore, it is preferred for the compounds of formula IA that R³ is CH₃, C₂H₅, CH(CH₃)₂, CH₂CH₂CH₃, CF₃, CHFCH₃, cyclopropyl, wherein the ring is substituted by halogen, or CN, and that R⁴ is C₁-C₄-alkyl, or that R³ and R⁴ together form C₅-C₆-cycloalkyl, which is unsubstituted, or partially or fully substituted by halogen, or CN.

Particularly preferred compounds of formula I are compounds of formula IA, wherein the variables R¹, R³, and R⁴ correspond to one row of table I below. Said compounds are referred to as compounds I-1 to I-18 in accordance with each row of table I.

TABLE I

| No | R¹ | R³ | R⁴ |
|---|---|---|---|
| I-1 | CH₃ | CH₃ | CH₃ |
| I-2 | CH₃ | CF₃ | CH₃ |
| I-3 | CH₃ | CH(CH₃)₂ | CH₃ |
| I-4 | CH₃ | 1-CN—c-C₃H₄ | CH₃ |
| I-5 | CH₃ | CHFCH₃ | CH₃ |

| | | |
|---|---|---|
| I-6 | CH₃ | CH₂CH₂CF₂CH₂CH₂ |

| | | | |
|---|---|---|---|
| I-7 | CH₂CH₃ | CH₃ | CH₃ |
| I-8 | CH₂CH₃ | CF₃ | CH₃ |
| I-9 | CH₂CH₃ | CH(CH₃)₂ | CH₃ |
| I-10 | CH₂CH₃ | 1-CN—c-C₃H₄ | CH₃ |
| I-11 | CH₂CH₃ | CHFCH₃ | CH₃ |

| | | |
|---|---|---|
| I-12 | CH₂CH₃ | CH₂CH₂CF₂CH₂CH₂ |

| | | | |
|---|---|---|---|
| I-13 | CH₂OCH₃ | CH₃ | CH₃ |
| I-14 | CH₂OCH₃ | CF₃ | CH₃ |
| I-15 | CH₂OCH₃ | CH(CH₃)₂ | CH₃ |
| I-16 | CH₂OCH₃ | 1-CN—c-C₃H₄ | CH₃ |
| I-17 | CH₂OCH₃ | CHFCH₃ | CH₃ |

| | | |
|---|---|---|
| I-18 | CH₂OCH₃ | CH₂CH₂CF₂CH₂CH₂ |

Especially preferred compounds of formula I are compounds of formula IA, wherein the variables R¹, R³, and R⁴ correspond to one row of table la below. Said compounds are referred to as compounds I-1 to I-17 in accordance with each row of table la.

TABLE Ia

| No | R¹ | R³ | R⁴ |
|---|---|---|---|
| I-1 | CH₃ | CH₃ | CH₃ |
| I-2 | CH₃ | CF₃ | CH₃ |
| I-3 | CH₃ | CH(CH₃)₂ | CH₃ |
| I-4 | CH₃ | 1-CN—c-C₃H₄ | CH₃ |
| I-5 | CH₃ | CHFCH₃ | CH₃ |
| I-7 | CH₂CH₃ | CH₃ | CH₃ |
| I-8 | CH₂CH₃ | CF₃ | CH₃ |
| I-9 | CH₂CH₃ | CH(CH₃)₂ | CH₃ |
| I-10 | CH₂CH₃ | 1-CN—c-C₃H₄ | CH₃ |
| I-11 | CH₂CH₃ | CHFCH₃ | CH₃ |
| I-13 | CH₂OCH₃ | CH₃ | CH₃ |
| I-14 | CH₂OCH₃ | CF₃ | CH₃ |
| I-15 | CH₂OCH₃ | CH(CH₃)₂ | CH₃ |
| I-16 | CH₂OCH₃ | 1-CN—c-C₃H₄ | CH₃ |
| I-17 | CH₂OCH₃ | CHFCH₃ | CH₃ |

With regard to the compounds I-2, I-3, I-4, I-5, I-8, I-9, I-10, I-11, I-14, I-15, I-16, I-17, it is to be understood, as explained above, that the compounds may be present in two enantiomeric forms, which are all understood to be encompassed by the present invention, either in isolated form or as a mixture.

The chemical formulae of the compounds b5, b6, b7, b8, b9, b10, b11, b12, b13, b14, b15, b16, b17, and b18, are depicted below.

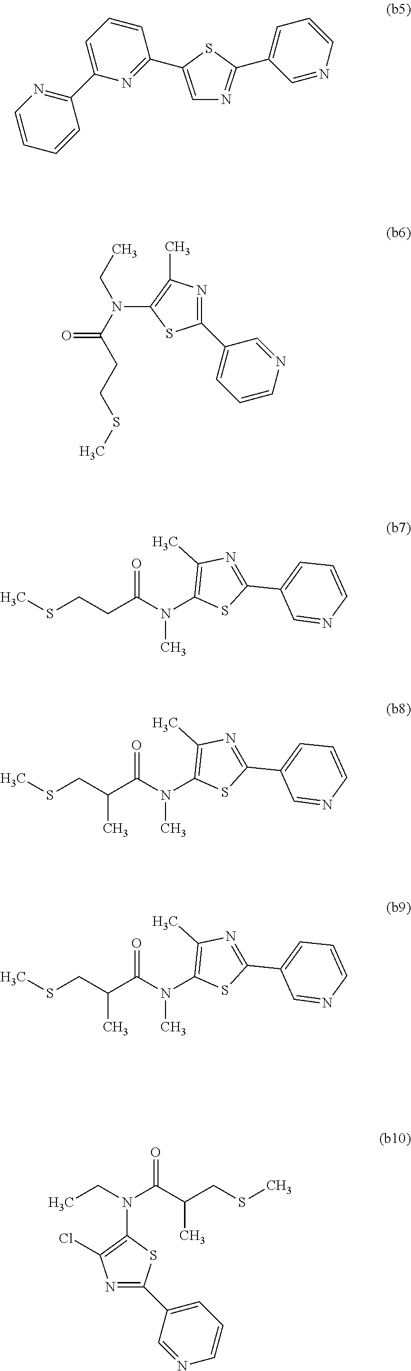 [SMILES: c1ccc(-c2cccc(-c3cnc(-c4cccnc4)s3)n2)nc1 ; CCN(C(=O)CCSC)c1sc(-c2cccnc2)nc1C ; CSCCC(=O)N(C)c1sc(-c2cccnc2)nc1C ; CSCC(C)C(=O)N(C)c1sc(-c2cccnc2)nc1C ; CSCC(C)C(=O)N(C)c1sc(-c2cccnc2)nc1C ; CCN(C(=O)C(C)CSC)c1sc(-c2cccnc2)nc1Cl]

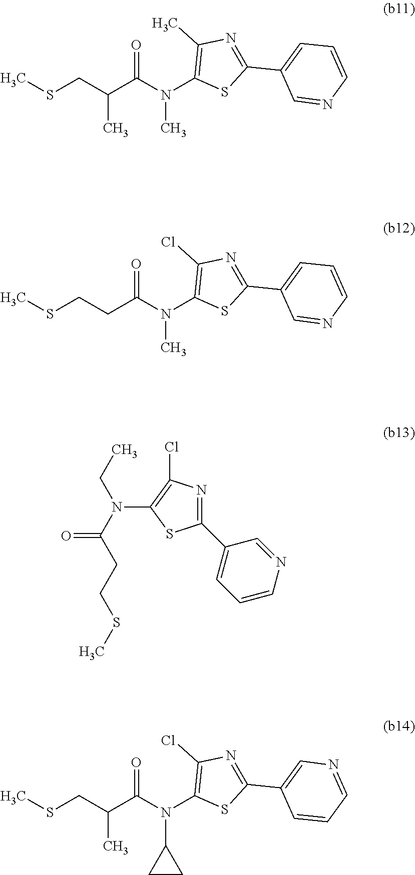 [SMILES: CSCC(C)C(=O)N(C)c1sc(-c2cccnc2)nc1C ; CSCCC(=O)N(C)c1sc(-c2cccnc2)nc1Cl ; CCN(C(=O)CCSC)c1sc(-c2cccnc2)nc1Cl ; CSCC(C)C(=O)N(c1sc(-c2cccnc2)nc1Cl)C1CC1]

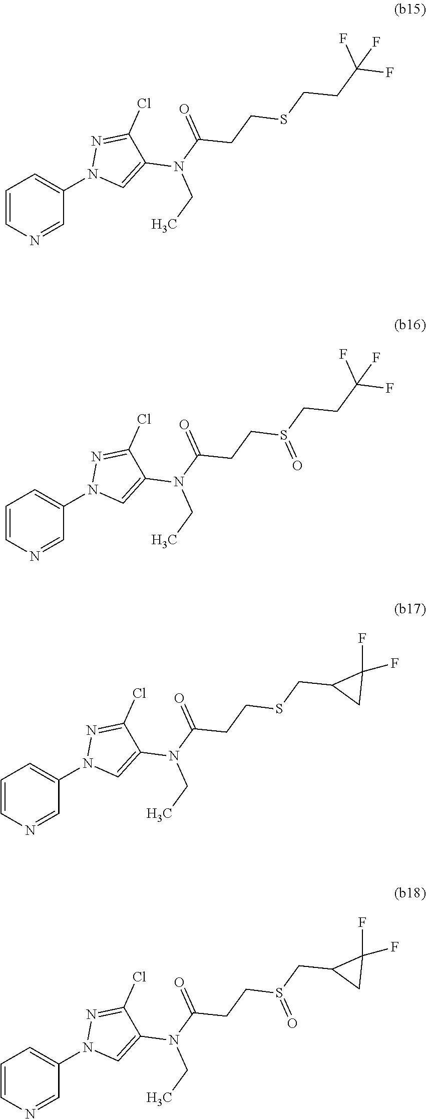 [SMILES: CCN(C(=O)CCSCCC(F)(F)F)c1cn(-c2cccnc2)nc1Cl ; CCN(C(=O)CCS(=O)CCC(F)(F)F)c1cn(-c2cccnc2)nc1Cl ; CCN(C(=O)CCSCC1CC1(F)F)c1cn(-c2cccnc2)nc1Cl ; CCN(C(=O)CCS(=O)CC1CC1(F)F)c1cn(-c2cccnc2)nc1Cl]

In preferred embodiments in connection with the uses and methods of the invention comprising the application of a pesticide (b) as defined herein, as well as in connection with the compositions as defined herein, and the uses and methods comprising the application of said composition, the pesticide (b) is a compound selected from the group consisting of b4, b5, b10, b11, and b14, preferably from b5, b10, b11, and b14. In other preferred embodiments, the pesticide (b) is a compound selected from the group consisting of b15, b16, b17, and b18. In other preferred embodiments, the pesticide (b) is a compound selected from the group consisting of b5, b10, b11, b14, b15, b16, b17, and b18.

As indicated above, it has been found that a pesticide (a), i.e. a compound of formula I, or a pesticide (b), i.e. a compound selected from b1, b2, b3, b4, b5, b6, b7, b8, b9, b10, b11, b12, b13, and b14, preferably from b5, b6, b7, b8, b9, b10, b11, b12, b13, and b14, or a compound selected from b5, b6, b7, b8, b9, b10, b11, b12, b13, b14, b15, b16, b17, and b18, or a compound selected from b15, b16, b17, and b18, may advantageously be used for protecting plants, the plant propagation material thereof, or soil or water, in which the plants are growing, against the attack or infestation by gastropods.

It is to be understood that the use for protecting plants, the plant propagation material thereof, or soil or water, in which the plants are growing, against the attack or infestation by gastropods, means that mollusc damage to plants can be reduced. This is achieved by either repelling the gastropods or killing the gastropods, e.g. if they ingest the pesticide (a) or (b) by feeding from a treated plant. The use therefore preferably comprises applying the pesticide (a) or (b) to the plants, the plant propagation material thereof, or soil or water, in which the plants are growing, in particular to the plants or the plant propagation material, especially to the seeds.

It is to be understood that the present invention also relates to a method for protecting plants, the plant propagation material thereof, or soil or water, in which the plants are growing, against the attack or infestation by gastropods comprising the application of a pesticide (a) or (b). It is to be understood that it is in each case also referred to such a method, when it is referred to a use in the following.

In one embodiment, the present invention relates to the use of a pesticide (a), i.e. a compound of formula I as defined above, for protecting plants, the plant propagation material thereof, or soil or water, in which the plants are growing, against the attack or infestation by gastropods.

In a preferred embodiment, the present invention relates to the use of a compound of formula IA as defined above, for protecting plants, the plant propagation material thereof, or soil or water, in which the plants are growing, against the attack or infestation by gastropods.

In a more preferred embodiment, the present invention relates to the use of compound selected from compounds I-1, I-2, I-3, I-4, I-5, I-6, I-7, I-8, I-9, I-10, I-11, I-12, I-13, I-14, I-15, I-16, I-17, and I-18, preferably from I-1, I-2, I-3, I-4, I-5, I-7, I-8, I-9, I-10, I-11, I-13, I-14, I-15, I-16, and I-17, for protecting plants, the plant propagation material thereof, or soil or water, in which the plants are growing, against the attack or infestation by gastropods.

In another embodiment, the present invention relates to the use of a pesticide (b), i.e. a compound selected from compounds b1, b2, b3, b4, b5, b6, b7, b8, b9, b10, b11, b12, b13, and b14, preferably from b5, b6, b7, b8, b9, b10, b11, b12, b13, and b14, or a compound selected from b5, b6, b7, b8, b9, b10, b11, b12, b13, b14, b15, b16, b17, and b18, or a compound selected from b15, b16, b17, and b18 as defined above, for protecting plants, the plant propagation material thereof, or soil or water, in which the plants are growing, against the attack or infestation by gastropods.

In a more preferred embodiment, the present invention relates to the use of a compound selected from compounds b4, b5, b10, b11, and b14.

As indicated above, it has further been found that a pesticide (a), i.e. a compound of formula I, or a pesticide (b), i.e. a compound selected from b1, b2, b3, b4, b5, b6, b7, b8, b9, b10, b11, b12, b13, and b14, preferably from b5, b6, b7, b8, b9, b10, b11, b12, b13, and b14, or a compound selected from b5, b6, b7, b8, b9, b10, b11, b12, b13, b14, b15, b16, b17, and b18, or a compound selected from b15, b16, b17, and b18, may advantageously be applied in a method for controlling gastropods, which comprises contacting the gastropods or their food supply, habitat or breeding ground; or plants or plant propagation material thereof, or soil or water, wherein the gastropods are present or may occur, with a molluscicidally effective amount of a pesticide (a) or (b).

The term “controlling gastropods” is to be understood as achieving an observable effect on growth, including the effects of necrosis, death, retardation, prevention, removal, destruction, or otherwise diminishing the occurrence and activity of the target organism. Preferably, control includes paralysis, repellency, feeding inhibition or killing of the gastropods. Particularly preferably, control of the gastropods is achieved by killing the gastropods, after they have been in contact with or have ingested the pesticide (a) or (b).

In connection with the control of gastropods, it is typically required that the pesticide (a) or (b) is applied in a molluscicidally effective amount, i.e. in an amount, which is sufficient to ensure control of gastropods. The molluscicidally effective amount can vary for the various compounds/compositions used according to the invention. A molluscicidally effective amount of the compounds of the invention will also vary according to the prevailing conditions such as desired molluscicidal effect and duration, weather, target species, locus, mode of application, and the like.

As used herein, the term “contacting with” means applying the pesticide (a) or (b) to something as indicated, or treating something as indicated with the pesticide (a) or (b).

It is to be understood that the present invention also relates to the use of a pesticide (a) or (b) for controlling gastropods, wherein the pesticide (a) or (b) is contacted with the gastropods or their food supply, habitat or breeding ground; or plants or plant propagation material thereof, or soil or water, wherein the gastropods are present or may occur, in a molluscicidally effective amount. It is to be understood that it is in each case also referred to such a use, when it is referred to a method in the following.

In one embodiment, the present invention relates to a method for controlling gastropods, which comprises contacting the gastropods or their food supply, habitat or breeding ground; or plants or plant propagation material thereof, or soil or water, wherein the gastropods are present or may occur, with a molluscicidally effective amount of a pesticide (a), i.e. a compound of formula I as defined above.

In a preferred embodiment, the present invention relates to a method for controlling gastropods, which comprises contacting the gastropods or their food supply, habitat or breeding ground; or plants or plant propagation material thereof, or soil or water, wherein the gastropods are present or may occur, with a molluscicidally effective amount of a compound of formula IA as defined above.

In a more preferred embodiment, the present invention relates to a method for controlling gastropods, which comprises contacting the gastropods or their food supply, habitat or breeding ground; or plants or plant propagation material thereof, or soil or water, wherein the gastropods are present or may occur, with a molluscicidally effective amount of a compound selected from compounds I-1, I-2, I-3, I-4, I-5, I-6, I-7, I-8, I-9, I-10, I-11, I-12, I-13, I-14, I-15, I-16, I-17, and I-18, preferably from I-1, I-2, I-3, I-4, I-5, I-7, I-8, I-9, I-10, I-11, I-13, I-14, I-15, I-16, and I-17.

In another embodiment, the present invention relates to a method for controlling gastropods, which comprises contacting the gastropods or their food supply, habitat or breeding ground; or plants or plant propagation material thereof, or soil or water, wherein the gastropods are present or may occur, with a molluscicidally effective amount of a compound selected from compounds b1, b2, b3, b4, b5, b6, b7, b8, b9, b10, b11, b12, b13, and b14, preferably from b5, b6, b7, b8, b9, b10, b11, b12, b13, and b14, or a compound selected from b5, b6, b7, b8, b9, b10, b11, b12, b13, b14, b15, b16, b17, and b18, or a compound selected from b15, b16, b17, and b18 as defined above.

In a preferred embodiment, the present invention relates to a method for controlling gastropods, which comprises contacting the gastropods or their food supply, habitat or breeding ground; or plants or plant propagation material thereof, or soil or water, wherein the gastropods are present or may occur, with a molluscicidally effective amount of a compound selected from compounds b4, b5, b10, b11, and b14 as defined above.

In connection with the above defined use and method according to the invention, the following gastropods, plants, mixing partners, formulations and application methods are particularly preferred. The preferred embodiments are to be understood as preferred on their own as well as preferably in combination with each other, and in combination with the preferred embodiments regarding the pesticide (a) or (b) as defined above. In particular, the preferred embodiments are to be understood as preferred in combination with any one of compounds I-1, I-2, I-3, I-4, I-5, I-6, I-7, I-8, I-9, I-10, I-11, I-12, I-13, I-14, I-15, I-16, I-17, or I-18, preferably any one of I-1, I-2, I-3, I-4, I-5, I-7, I-8, I-9, I-10, I-11, I-13, I-14, I-15, I-16, or I-17, as pesticide (a), or any one of compounds b1, b2, b3, b4, b5, b6, b7, b8, b9, b10, b11, b12, b13, or b14, preferably any one of b5, b6, b7, b8, b9, b10, b11, b12, b13, or b14, or any one of b5, b6, b7, b8, b9, b10, b11, b12, b13, b14, b15, b16, b17, or b18, or any one of b15, b16, b17, or b18 as pesticide (b).

In connection with the following preferred embodiments regarding inter alia gastropods, plants, mixing partners, formulations, and application methods, it is in each case referred to the “use or method according to the invention comprising the application of a pesticide (a) or (b) as defined above” or to the “use or method according to the invention” to indicate that the preferences refer to the above defined use or method in connection with the protection from gastropods or the control of gastropods, respectively, wherein the pesticide (a) or (b) as defined above is used or applied.

As used herein, the term “gastropod” includes the three sub-classes Prosobranchia, Opistho-branchia and Pulmonata. In particular, the term “gastropods” includes Achatina spp., Agriolimax spp., Arion spp. (e.g. A. ater, A. circumscriptus, A. distinctus, A. fasciatus, A. hortensis, A. intermedius, A. rufus, A. subfuscus, A. silvaticus, A. lusitanicus), Biomphalaria spp., Bradybaena spp. (e.g. B. fruticum), Bulinus spp., Cantareus spp. (e.g. C. asperses), Cepaea spp. (e.g. C. hortensis, C. nemoralis), Cernuella spp., Cochlicella spp., Cochlodina spp. (e.g. C. laminata), Deroceras spp. (e.g. D. agrestis, D. empiricorum, D. laeve, D. panornimatum, D. reticulatum), Discus spp. (e.g. D. rotundatus), Euomphalia spp., Galba spp. (e.g. G. trunculata), Helicella spp. (e.g. H. itala, H. obvia), Helicigona spp. (e.g. H. arbustorum), Helicodiscus spp., Helix spp. (e.g. H. aperta, H. aspersa, H. pomatia), Limax spp. (e.g. L. cinereoniger, L. flavus, L. marginatus, L. maximus, L. tenellus), Lymnaea spp. (e.g. L. stagnalis), Milax spp. (e.g. M. gagates, M. marginatus, M. sowerbyi, M. budapestensis), Oncomelania spp., Opeas spp., Oxyloma spp. (e.g. O. pfeifferi), Pomacea spp. (e.g. P. canaliculata), Succinea spp., Tandonia spp. (e.g. T. budapestensis, T. sowerbyi), Theba spp., Vallonia spp., and Zonitoides spp. (e.g. Z. nitidus).

In one embodiment of the use or method according to the invention, the gastropods are selected from the group consisting of Achatina spp., Agriolimax spp., Arion spp., Biomphalaria spp., Bradybaena spp., Bulinus spp., Cantareus spp., Cepaea spp., Cernuella spp., Cochlicella spp., Cochlodina spp., Deroceras spp., Discus spp., Euomphalia spp., Galba spp., Helicella spp., Helicigona spp., Helicodiscus spp., Helix spp., Limax spp., Lymnaea spp., Milax spp., Oncomelania spp., Opeas spp., Oxyloma spp., Pomacea spp., Succinea spp., Tandonia spp., Theba spp., Vallonia spp., and Zonitoides spp.

In a preferred embodiment of the use or method according to the invention, the gastropods are selected from the group consisting of Achatina spp., Agriolimax spp., Arion ater, Arion hortensis, Arion lusitanicus, Bradybaena spp., Cantareus spp., Cepaea spp., Cernuella spp., Cochlicella spp., Cochlodina spp., Deroceras agreste, Deroceras reticulatum, Discus spp., Euomphalia spp., Helicella spp., Helicigona spp., Helicodiscus spp., Helix spp., Limax spp., Milax spp., Opeas spp., Oxyloma spp., Tandonia spp., Theba spp., Vallonia spp., and Zonitoides spp.

In a more preferred embodiment of the use or method according to the invention, the gastropods are selected from the group consisting of Agriolimax spp., Arion ater, Arion hortensis, Arion lusitanicus, Helix spp., Limax spp., Milax spp., Deroceras agreste, and Deroceras reticulatum, particularly preferably from the group consisting of Arion ater, Arion hortensis, Arion lusitanicus, Deroceras agreste, and Deroceras reticulatum.

In one particularly preferred embodiment of the use or method according to the invention, the gastropods are Achatina spp. (G-1), especially Achatina fulica (G-1a). In one particularly preferred embodiment of the use or method according to the invention, the gastropods are Agriolimax spp. (G-2). In one particularly preferred embodiment of the use or method according to the invention, the gastropods are Arion spp. (G-3), especially Arion ater (G-3a), Arion hortensis (G-3b), or Arion lusitanicus (G-3c). In another particularly preferred embodiment of the use or method according to the invention, the gastropods are Cernuella spp. (G-4), especially Cernuella virgata (G-4a). In another particularly preferred embodiment of the use or method according to the invention, the gastropods are Cochlicella spp. (G-5), especially Cochlicella acuta (G-5a) or Cochlicella barbara (G-5b). In another particularly preferred embodiment of the use or method according to the invention, the gastropods are Deroceras spp. (G-6), especially Deroceras agreste (G-6a) or Deroceras reticulatum (G-6b). In another particularly preferred embodiment of the use or method according to the invention, the gastropods are Helix spp. (G-7), especially Cryptomphalus aspersus (formerly Helix aspersa) (G-7a). In another particularly preferred embodiment of the use or method according to the invention, the gastropods are Limax spp. (G-8), especially Limax flavus (G-8a) or Limax maximus (G-8b). In another particularly preferred embodiment of the use or method according to the invention, the gastropods are Milax spp. (G-9), especially Milax gagates (G-9a). In another particularly preferred embodiment of the use or method according to the invention, the gastropods are Pomocea spp. (G-10), especially Pomocea canaliculata (G-10a). In another particularly preferred embodiment of the use or method according to the invention, the gastropods are Tandonia spp. (G-11), especially Tandonia budapestensis (G-11a). In another particularly preferred embodiment of the use or method according to the invention, the gastropods are Theba spp. (G-12), especially Theba pisana (G-12a). In another particularly preferred embodiment of the use or method according to the invention, the gastropods are Veronicellidae (G-13).

As used herein, the term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including wild type plants and plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, cry1Ab, bar, cry1Fa2, cry1Ac, cry34Ab1, cry35AB1, cry3A, cryF, cry1F, mcry3a, cry2Ab2, cry3Bb1, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In one embodiment of the use or method according to the invention, the plants are selected from the group consisting of cereals including wheat, maize, barley, oat, rye, sorghum, and rice; oil crops including rapeseed and turnip rape, mustard, poppies, olives, sunflowers, coconuts, castor, cacao and peanuts; fiber plants including cotton, flax, hemp or jute; leaf vegetables including lettuce, spinach, and cabbage; bud vegetables including Brussels sprouts; tuber vegetables including potatoes; root vegetables including turnips, beets such as sugar or fodder beet, radishes, carrots, and swedes; leguminous vegetables including lentils, alfalfa, soybeans, beans and peas; stem shoot vegetables including asparagus; marrows including pumpkins, cucumbers and melons; laurels including avocado, cinnamonium and camphor; tobacco, nuts, coffee, egg plants, sugar cane, tea, pepper, grapevines, hops, the banana family, and latex plants; fruits including pome fruit, stone fruit, apples, pears, plums, peaches, almonds, cherries, strawberries, raspberries and blackberries; citrus fruits including oranges, lemons, grapefruits and tangerines; clover and newly sown leys; medicinal herbs; and ornamentals.

In a preferred embodiment of the use or method according to the invention, the plants are selected from the group consisting of cereals including wheat, maize, barley, oat, rye, and rice; cotton; oil crops including poppies, rapeseed and turnip rape; leaf vegetables including lettuce, spinach, and cabbage; bud vegetables including Brussels sprouts; tuber vegetables including potatoes; root vegetables including turnips, beets including sugar and fodder beets, radishes, carrots, and swedes; leguminous vegetables including alfalfa, beans and peas; stem shoot vegetables including asparagus; tobacco; fruits including strawberries; clover and newly sown leys; medicinal herbs; and ornamentals.

In a more preferred embodiment of the use or method according to the invention, the plants are selected from the group consisting of cereals (P-1), vegetables (P-2), fruits (P-3), oil crops (P-4), tobacco (P-5), and ornamental plants (P-6).

In one preferred embodiment of the use or method according to the invention, the plants are crop plants selected from the group consisting of cereals (P-1), vegetables (P-2), fruits (P-3), oil crops (P-4), and tobacco (P-5).

In a particularly preferred embodiment, the plants are crop plants selected from the group consisting of wheat, barley, maize, rice, lettuce, spinach, cabbage, Brussels sprout, potatoes, sugar beets, radishes, carrots, beans, peas, asparagus, strawberries, rapeseed, and poppies.

In one particularly preferred embodiment of the use or method according to the invention, the plants are cereals (P-1). Preferred cereals include wheat (P-1a), barley (P-1 b), maize (P-1c), and rice (P-1d). One especially preferred cereal is wheat (P-1a). Another especially preferred cereal is rice (P-1d).

In another particularly preferred embodiment of the use or method according to the invention, the plants are vegetables (P-2). Preferred vegetables include leaf vegetables, bud vegetables, tuber vegetables, root vegetables, leguminous vegetables, and stem shoot vegetables.

Particularly preferred vegetables include lettuce (P-2a), spinach (P-2b), cabbage (P-2c), Brussels sprout (P-2d), potatoes (P-2e), sugar beets (P-2f), radishes (P-2g), carrots (P-2h), beans (P-2i), peas (P-2j), and asparagus (P-2k).

In another particularly preferred embodiment of the use or method according to the invention, the plants are fruits (P-3), especially strawberries (P-3a).

In another particularly preferred embodiment of the use or method according to the invention, the plants are oil crops (P-4), preferably rapeseed (P-4a) or poppies (P-4b), especially rapeseed (P-4a). Rapeseed (Brassica napus) is also known as rape, oilseed rape, rapa, rappi, rapaseed (and, in the case of one particular group of cultivars, canola).

In another particularly preferred embodiment of the use or method according to the invention, the plants are tobacco plants (P-5).

In another preferred embodiment, the plants are ornamental plants (P-6). Preferred ornamental species include: Ageratum, Alonsoa, Anemone spp., Anisodontea capsenisis, Anthemis, Antirrhinum, Rhododendron spp., Begonia spp. (eg. B. elatior, B. semperflorens, B. tubereux), Bougainvillea spp., Brachycome spp., Calceolaria, Capsicum annuum, Catharanthus roseus, Ornamental Brassica, Canna spp., Chrysanthemum, Cineraria spp. (C. maritime), Crassula coccinea, Cuphea ignea, Dicentra spectabilis, Dorotheantus, Eustoma grandiflorum, Forsythia, Fuchsia spp., Geranium Gnaphalium, Gomphrena globosa, Heliotropium, Helianthus, Hibiscus, Hortensia, Hosta, Hypoestes phyllostachya, Impatiens spp. (I. Walleriana), Iresines, Kalanchoe spp., Lantana camara, Lavatera trimestris, Leonotis leonurus, Lilium, Mesembryanthemum, Mimulus, Nemesia, Tagetes, Dianthus spp. (carnation), Canna, Oxalis, Bellis, Pelargonium spp. (P. peltatum, P. Zonale), Viola spp. (pansy), Petunia, Plecthranthus, Poinsettia, Parthenocissus spp. (P. Quinquefolia, P. Tricuspidata), Primula, Ranunculus, Rosa spp. (rose), Salvia, Scaevola aemola, Schizanthus wisetonensis, Solanum, Surfinia, Tagetes spp., Nicotinia, Verbena spp., Zinnia spp. and other bedding plants. Preferred within this class of ornamental crops are Viola, Petunia, Begonia, Impatiens, Geranium (including from seeds and cuttings), Chrysanthemum (including from cuttings), Rosa (including pot plants and from cuttings), Poinsettia, Ranunculus, Fuchsia, Salvia and Hortensia.

In connection with the above defined use or method according to the invention, the compounds of the invention may either be applied alone or in combination with an additional molluscicide.

In one embodiment of the use or method according to the invention, the pesticide (a) or (b) is applied alone, i.e. not in combination with a further active agent, in particular not with an additional pesticide.

In one embodiment of the use or method according to the invention, the pesticide (a) is not applied in combination with an insecticide.

In a preferred embodiment of the use or method according to the invention, the pesticide (a) is not applied in combination with Ryanodine receptor-modulator from the class of diamide compounds.

In a preferred embodiment of the use or method according to the invention, the pesticide (a) is not applied in combination with an anthranilamide compound, in particular not with a N-thio-anthranilamide compound with a sulfimine or sulfoximine group as, e.g., described in WO2007/006670, WO2013/024009, WO2013/024010 and WO2013/174645. In a more preferred embodiment, the pesticide (a) is not applied in combination with a compound selected from M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; M.28.5b) N-[4-chloro-2-[(diethyl-lambda-4-sulfanyl idene)carbamoyl]-6-methyl-phenyl]-2-(3-chloro-2-pyridyl)-5-(trifluoromethyl)pyrazole-3-carboxamide; M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; M.28.5d) N-[4,6-dichloro-2-[(di-2-propyl-lambda-4-sulfanylidene)carbamoyl]-phenyl]-2-(3-chloro-2-pyridyl)-5-(trifluoro-methyl)pyrazole-3-carboxamide; and M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide.

In another more preferred embodiment of the use or method according to the invention, the pesticide (a) is not applied in combination with tetraniliprole.

In another preferred embodiment of the use or method according to the invention, the pesticide (a) is not applied in combination with broflanilide.

In another preferred embodiment of the use or method according to the invention, the pesticide (a) is not applied in combination with tioxazafen.

Thus, it is preferred according to the invention that the pesticide (a) is not applied in combination with an insecticide, in particular not with an insecticide selected from the group consisting of anthranilamide compounds including N-thio-anthranilamide compounds with a sulfimine or sulfoximine group as indicated above and tetraniliprole, broflanilide and tioxazafen.

In one embodiment of the use or method according to the invention, the pesticide (a) is not applied in combination with a fungicide.

In one embodiment of the use or method according to the invention, the pesticide (a) is not applied in combination with a biopesticide.

Biopesticides have been defined as a form of pesticides based on micro-organisms (bacteria, fungi, viruses, nematodes, etc.) or natural products (compounds, such as metabolites, proteins, or extracts from biological or other natural sources) (U.S. Environmental Protection Agency: http://www.epa.gov/pesticides/biopesticides/). Biopesticides fall into two major classes, microbial and biochemical pesticides:

(1) Microbial pesticides consist of bacteria, fungi or viruses (and often include the metabolites that bacteria and fungi produce). Entomopathogenic nematodes are also classified as microbial pesticides, even though they are multi-cellular.

(2) Biochemical pesticides are naturally occurring substances or structurally-similar and functionally identical to a naturally-occurring substance and extracts from biological sources that control pests or provide other crop protection uses as defined below, but have non-toxic mode of actions (such as growth or developmental regulation, attractants, repellents or defence activators (e.g. induced resistance) and are relatively non-toxic to mammals.

In a preferred embodiment of the invention, the pesticide (a) is thus not applied in combination with an insecticide, a fungicide or a biopesticide.

In one embodiment of the use or method according to the invention, the pesticide (a) or (b) is applied in combination with at least one additional molluscicide. As used herein, the term “molluscicide” is to be understood as a compound, which is suitable for protecting plants, the plant propagation material thereof, or soil or water, in which the plants are growing, against the attack or infestation by gastropods, and/or which is suitable for controlling gastropods by contacting the gastropods or their food supply, habitat or breeding ground; or plants or plant propagation material thereof, or soil or water, wherein the gastropods are present or may occur, with a molluscicidally effective amount thereof.

If the pesticide (a) is applied in combination with at least one molluscicide, it is preferred that the combination of active agents is not applied together with an additional pesticide, in particular not with an additional fungicide, insecticide or biopesticide. It is preferred that a binary combination of pesticide (a) and an additional molluscicide is applied, i.e. that only one molluscicide is applied in combination with pesticide (a).

In one preferred embodiment, the pesticide (a) or (b) and the at least one additional molluscicide are applied in the form of a mixture. Preferred are binary mixtures of one compound of the present invention as component I with one additional molluscicide as defined hereinafter as component II. Preferred weight ratios for such binary mixtures are from 5000:1 to 1:5000, preferably from 1000:1 to 1:1000, more preferably from 100:1 to 1:100, particularly preferably from 10:1 to 1:10. The relative amounts of each compound in the mixture may be adjusted to take account of the properties of the selected compounds themselves, and to maximize the molluscicidal effect.

In another preferred embodiment, the pesticide (a) or (b) and the additional molluscicide are applied separately. In particular, the pesticide (a) or (b) may be applied to the plants or plant propagation material thereof, preferably to the seeds of the plants, or to the foliage of the plants, or to the soil around the base of a plant stem, in particular around the seedling stem, and the at least one additional molluscicide may be applied in the form of a bait composition. The gastropods will then be repelled by the pesticide (a) or (b) and instead be attracted and killed by the bait composition.

In connection with the combined application of a pesticide (a) or (b) and at least one additional molluscicide, it is to be understood that also a combination of the pesticide (a) and the pesticide (b) may be applied. In one embodiment, the pesticide (a) is I-1 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-2 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-3 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-4 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-5 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-6 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-7 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-8 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-9 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-10 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-11 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-12 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-13 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-14 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-15 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-16 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-17 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18. In one embodiment, the pesticide (a) is I-18 and the pesticide (b) is any one of b1, b2, b3, b4, b5, b6, b7, b8, b9, b10, b11, b12, b13, or b14, or any one of b5, b6, b7, b8, b9, b10, b11, b12, b13, b14, b15, b16, b17, or b18.

In one embodiment of the use or method according to the invention, the pesticide (a) or (b) is applied in combination with at least one molluscicide selected from the group consisting of metaldehyde (M-1), methiocarb (M-2), thiodicarb (M-3), spinosad (M-4), spinetoram (M-5), spinosyn mixture (M-6), niclosamine (M-7), ferric phosphate (M-8), fentin hydroxide (M-9), fentin acetate (M-10), tazimcarb (M-11), trifenmorph (M-12), trimethacarb (M-13), bensultap (M-14), pymetrozin (M-15), a strobilurin compound (M-16), or metallic ions (M-17), such as iron (M-17a) or copper (M-17b) or aluminum (M-17c), for example in the form of iron phosphate or iron chelate (such as FeEDTA).

In a preferred embodiment of the use or method according to the invention, the pesticide (a) or (b) is applied in combination with at least one molluscicide selected from the group consisting of metaldehyde, niclosamine, ferric phosphate, fentin hydroxide, fentin acetate, tazimcarb, trifenmorph, trimethacarb, bensultap, and pymetrozin.

In a further preferred embodiment, the molluscicide is spinetoram, spinosyn mixture, spinosad, methiocarb or thiodicarb. If the pesticide (a) is applied in combination with a molluscicide, which is spinetoram, spinosyn mixture, spinosad, methiocarb or thiodicarb, it is preferred that a binary combination of these active agents is applied, i.e. that the combination of active agents is not applied together with an additional pesticide, in particular not with an additional fungicide, insecticide or biopesticide.

In a further preferred embodiment, the molluscicide is not any one of spinetoram, spinosyn mixture, spinosad, methiocarb or thiodicarb, in particular not any one of spinosad, methiocarb or thiodicarb. For example, it is often desired to avoid methiocarb and thiodicarb, as they are highly toxic to mammals.

In a further preferred embodiment, the molluscicide is a strobilurin compound is selected from the group consisting of azoxystrobin, picoxystrobin, trifloxystrobin, kresoxim methyl, enestrobin, orysastrobin, dimoxystrobin, metominostrobin, pyraclostrobin, fluoxastrobin, famoxadone and fenamidone. More preferably the strobilurin compound is selected from the group consisting of azoxystrobin, picoxystrobin, trifloxystrobin, kresoxim methyl and pyraclostrobin. Most preferably, the strobilurin compound is azoxystrobin.

In a further preferred embodiment, the molluscicide is an iron ion, which is in the form of iron chelate, especially iron EDTA. Alternatively, the iron ion is in the form of iron phosphate.

In a further preferred embodiment, the molluscicide is a copper ion, preferably in the form of copper sulphate.

In a further preferred embodiment, the molluscicide is an aluminum ion, preferably in the form of aluminum sulphate.

In another embodiment of the use or method according to the invention, the pesticide (a) or (b) is applied in combination with at least one plant-based molluscicide selected from the group consisting of saponins (M-18), tannins (M-19), alkaloids, alkenyl phenols, glycoalkaloids, flavonoids, sesquiterpene lactons and terpenoids.

Of these, saponins appear to be the most potent class of molluscicides. Plants containing these compounds belong mostly to the plant families Euphorbiaceae, Phytolaccaceae, Polygonaceae, Rutaceae and particulary Mimosaceae, Papilionaceae and Caesalpiniaceae. The latter three families contain many easily cultivated, drought-resistant trees with high-quality seed proteins. Saponin is preferably obtained from a plant selected from the group consisting of tea, and/or yucca and/or quillaia and/or quinoa and/or soya, particularly preferably from tea. Furthermore, saponin can be obtained from Camellia oleifera, Gleditsia amorphoides, and Quillaja saponaria.

Saponin-based molluscicides may for example be used in the form of aqueous solutions of plant powders. Alternatively, water extracts of the plants may be used. An example in this regard is endod berries. Water extracts of these berries can be obtained by soaking the carefully crushed berries in water for 24 hours at concentrations of 150 mg/l, 75 mg/l, and 37.5 mg/l.

Also tannin-bearing plants are considered to have a potential as molluscicides. For example, water extracts of Krameria triandra, Hamamelis virginiana and Quercus spp. may be used. Other promising species are Camillia spp., Potentilla erecta, Alchemilla spp., Acacia catechu, Dalbergia nitidula, Arctostaphylus uva-ursi, and Chinchona succirubra.

In another embodiment, the at least one additional molluscicide is tannic acid, tea tannin, polyphenyl hydrolysis products thereof or a mixture thereof.

Plant molluscicides such as saponins may also be used in combination with a further component selected from the group consisting of carvacrol, thymol, eugenol, and methol. The combination of saponin with carvacrol is particularly preferred. Compositions comprising saponin and carvacrol typically comprise 0.1-10 wt-%, preferably 0.15-8 wt-%, more preferably 0.2-5 wt-% of saponin, and 0.1-10% wt-%, preferably 0.15-8 wt-% and more preferably 0.2-5 wt-% of carvacrol.

Furthermore, combinations of saponins and tannins are preferred.

Suitable plant molluscicide compositions are also described in EP2002721A2, which is incorporated by reference.

In another embodiment of the use or method according to the invention, the pesticide (a) or (b) is applied in combination with afidopyropen (M-20) as additional molluscicide. Afidopyropen has the IUPAC name [(3S,4R,4aR,6S,6aS,12R,12aS,12bS)-3-(cyclopropylcarbonyloxy)-1,2,3,4,4a,5,6,6a,12a,12b-decahydro-6,12-dihydroxy-4,6a,12b-trimethyl-11-oxo-9-(3-pyridyl)-11H,12H-benzo[f]pyrano[4,3-b]chromen-4-yl]methyl cyclopropanecarboxylate.

In one embodiment of the use or method according to the invention, the pesticide (a) or (b) is applied in combination with at least one molluscicide selected from M-1, M-2, M-3, M-4, M-5, M-6, M-7, M-8, M-9, M-10, M-11, M-12, M-13, M-14, M-15, M-16, M-17, M-18, M-19, and M-20 as defined above.

The compounds of the present invention are preferably applied in the form of an agricultural composition comprising an auxiliary and at least one compound of the present invention or a mixture thereof.

The compounds of the present invention or the mixtures thereof can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Mono-graph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides.

Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide.

Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e. g. components comprising compounds of the present invention and/or mixing partners as defined above, can be applied jointly (e.g. after tank mix) or consecutively.

The compounds of the present invention can be applied as such or in form of compositions comprising them as defined above. Furthermore, the compounds of the present invention can be applied together with a molluscicide as mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The compounds of the present invention are effective through both contact and ingestion. Furthermore, the compounds of the present invention can be applied to any and all developmental stages, such as egg, juvenile, and adult.

The application can be carried out both before and after the infestation of the crops, plants, plant propagation materials, such as seeds, soil, or the area, material or environment by the gastropod pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Soil drenching is particularly preferred according to the invention. During a soil drench, the diluted active agent(s) is/are poured over the roots of the plants near the stem or trunk. The active agent(s) then preferably disperse(s) within the plant and act systemically. Seed treatment techniques include seed dressing, seed coating, seed dusting, seed soaking, and seed pelleting. Seed treatment is also particularly preferred according to the invention and is described in further detail below. In furrow applications typically include the steps of making a furrow in cultivated land, seeding the furrow with seeds, applying the molluscicidally active compound to the furrow, and closing the furrow. Foliar application refers to the application of the molluscicidally active compound to plant foliage, e.g. through spray equipment. Foliar application is also particularly preferred according to the invention. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds, and the resulting seedling's roots and shoots against from gastropods. The present invention therefore also relates to a method for the protection of seeds from and of the seedling's roots and shoots from gastropods, said method comprising treating the seeds before sowing and/or after pregermination with a compound of the present invention. The protection of the seedling's roots and shoots is preferred.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/I Surfactant, 0 to 200 g/I antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/1) of the active ingredient, from 0.1 to 20% by weight (1 to 200 g/1) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the present invention may also be applied in the form of bait compositions, e.g. in the form of slug pellets.

In one aspect, the present invention therefore relates to a composition comprising
(i) at least one pesticide (a) or (b) as defined above;
(ii) at least one mollusc attractant; and optionally
(iii) at least one further additive.

The composition is suitable as a bait composition due to the presence of the mollusc attractant. The mollusc attractant is a substance, which attracts gastropods, e.g., due to a certain odor or taste, so that the gastropod gets in contact with or ingests the pesticide (a) or (b).

The bait composition may be applied in connection with the above defined uses and methods of the present invention. It is to be understood that the preferred embodiments regarding gastropods, plants, and mixing partners are also preferred in connection with the above defined bait composition. In particular, reference is made to the preferred embodiments defined in tables II, III, and IV.

In a preferred embodiment, the bait composition is applied in methods and uses according to the invention, wherein the plants and gastropods are defined according to any one of the rows of tables II or III.

In another preferred embodiment, the bait composition comprises at least one additional molluscicide. Preferably, the molluscicide is selected from the molluscicides defined above. In particular, active agent combinations according to any one of the rows of table IV are preferred.

Thus, the present invention relates in one embodiment to the use or method according to the invention as defined above, wherein the pesticide (a) or (b) is applied in the form of a compositions as defined above.

Of particular relevance in connection with the above defined compositions is the mollusc attractant, which causes the gastropods to get in contact with or ingest the pesticide (a) or (b).

In a preferred embodiment of the above defined composition, the mollusc attractant is

(iia) a phagostimulant, which is selected from the group consisting of ground cereals including cereal flours, cereal brans, and rice starch; crushed soybeans; crushed rapeseed; fish meal; molasses; and any combination thereof; or

(iib) an attractant, which is selected from the group consisting of plant materials including grass and grass seeds; extracts from fruits, cabbage, herbs, tomatoes or potatoes; beer; yeast; extracts from dead slugs; and any combination thereof; or

(iic) a combination of (iia) and (iib).

In a more preferred embodiment, the mollusc attractant is

(iia) a phagostimulant, which is selected from the group consisting of wheat flour, barley flour, rye flour, rice starch, and combinations thereof.

Phagostimulants may also advantageously act as carriers, so that an additional carrier is not necessarily required to form, e.g., slug pellets. However, mixtures of phagostimulants with other suitable organic and/or inorganic carriers may also be present in the compositions of the invention.

To make the composition more palatable for gastropods, one or more of the following substances may also be used as additive: a) vitamin B, in particular B1, B2, nicotinic acid or nicotinamide; b) vitamin E; c) animal or vegetable proteinaceous material, for example albumins and their hydrolytic degradation products, in particular those obtained by enzymatic hydrolysis by, for example, pepsin (such as metaproteins, proteoses, peptones, polypeptides, peptides, diketopiperazines and amino acids); d) one or more amino acids or salts or amides thereof, which may also be synthetic products; e) a nucleic acid or a hydrolytic degradation product thereof, such as a nucleotide, a nucleoside, adenine, guanine, cytosine, uracil or thymine; f) urea or carbamic acid; g) an ammonium salt, for example ammonium acetate; h) an amino sugar, for example glucosamine or galactosamine; i) compounds of sodium, potassium, calcium or magnesium, or traces of compounds of manganese, copper, iron, cobalt, zinc, aluminium, boron or molybdenum; j) phosphoric acid or sugar phosphates; k) water.

Furthermore, the composition may comprise at least one additive selected from bittering agents and repellents to protect farm animals, pets, and livestock, in particular hedgehogs and birds. The bittering agents and/or repellents will stop these animals from ingesting the composition due to a bitter taste and/or due to a certain type of repellency, e.g., due to a certain odor. In one embodiment, the bait composition may thus preferably comprise one or more bird repellents, such as anthraquinone. In another embodiment, the bait composition may thus preferably comprise one or more bittering agents, preferably denatonium benzoate and/or denatonium saccharide.

The bait composition may optionally comprise stabilizers that have a fungistatic, fungicidal, bacteriostatic and/or bactericidal action, such as sodium benzoate, methyl p-hydroxy-benzoate, cetyltrimethylammonium bromide, citric acid, tartaric acid, sorbic acid, phenols, alkylphenols or chlorinated phenols.

The composition may be formulated to provide a slow or delayed release of the pesticide (a) or (b) over time, so as to provide long-term protection against gastropods. Suitable slow-release additives which may be employed in the formulation include, for example, resins (such as urea/formaldehyde resins), soybean meal, waxes, stearates and oils (such as castor oil).

Other additives that may be used in the composition of the present invention include, for example, binders (such as methylcellosolve, polyvinylpyrrolidone, polyvinyl alcohol, polyacrylates, polymethacrylates, natural waxes, chemically modified waxes and synthetic waxes, sugars, starch, alginates, agar, lignosulphonates and gum arabic), humectants (such as polyalcohols, for example sugars or glycerol), preservatives, colorants and repellents for warm-blooded species.

The bait composition may also be coated to protect it from moisture degradation, and subsequent leaching of the pesticide (a) or (b) into the soil. Such a coating may extend the life of the bait composition, and reduce the re-application frequency needed. Suitably the bait composition does not prematurely degrade when it is applied to damp soil.

The bait composition is typically provided in the form of granules or pellets. The size of the pellets is such that they can be readily consumed by the target gastropods to ensure ingestion. Typically, the pellets are from about 1 to about 5 mm in length.

Also from the bait compositions described herein, the pesticide (a) or (b) may be released as such that they act systemically in the plants as they may be taken up by plant roots and transported around the plant. The uptake of the compound by the plant from the soil has the added benefit that not only the gastropods feeding from the slug pellets are exposed to the pesticide (a) or (b), but also the gastropods feeding from the plants. The systemic action of the pesticide (a) or (b) in the plant will last longer than the molluscicidal action of the bait composition itself, as the bait composition will leach the active agent after a certain time, while the active agent is then taken up by the plants in the surrounding area.

Formulations that are particularly suitable for bait compositions according to the present invention are granules or pellets which comprise from 0 to 90% by weight of carrier material, from 0.01 to 20 wt % by weight of active ingredient (i.e. pesticide (a) or (b)), from 10 to 95% by weight of phagostimulant, from 0.5 to 25% by weight of binder, and optionally from 0 to 15% by weight of other additives. Suitably, the granules or pellets comprise from 0 to 70 wt % by weight of carrier material, from 1 to 10% by weight of active ingredient, from 25 to 90% by weight of phagostimulant, from 5 to 20% by weight of binder, and optionally from 0 to 15% by weight of other additives.

The application rate of the bait composition of the invention depends on the concentration of the pesticide (a) or (b) in the formulation. Typically, the concentration of the pesticide (a) or (b) will be adjusted so that the composition may be applied at a dose in the range from about 1 to 15 kg bait/ha. When the composition comprises from 0.01 to 20 wt % of active ingredient, this is equivalent to applying between about 0.1 g ai/ha and 3000 g ai/ha. To achieve optimal control, it is preferable that the bait composition is distributed as uniformly as possible between the plants. In an embodiment, the rates for pesticide (a) or (b) per ha are estimated to be between 1 g and 1 kg, preferably 10 g to 250 g, more preferably 50 g to 200 g, ai. In a preference, a bait is concentrated between 0.01 and 5, preferably between 0.5 and 2 wt % of the compound.

The compositions of the present invention are also suitable for the protection of plant propagation material, for example seed, such as fruits, tubers or kernels, from gastropods. The propagation material can be treated with a suitable composition prior to planting, for example by soaking, spraying or coating seed prior to sowing. Alternatively, the composition can be applied directly to the locus at which the propagation material is to be planted (for example onto the ground, into a seed furrow, or into pot plant growing media).

In a preferred embodiment, the bait composition is applied to the soil, wherein plants are growing or are intended to grow.

In another preferred embodiment, the pesticide (a) or (b) is applied to plant propagation material, such as seeds, at a rate of 0.1 to 100, preferably 1 to 60, more preferably 4 to 40, g/100 kg of seeds.

The present invention is further illustrated by the following examples.

### EXAMPLES

The activity of the compounds of formula I of the present invention could be demonstrated and evaluated in the following biological test.

### Example 1

Activity against Lehmannia valentia on cabbage; Whole plant assay by foliar spray treatment; Laboratory study:

The adult banded slug (Lehmannia valentia) was field-collected and maintained in plastic boxes lined with moist, unbleached, absorbent paper at a temperature of from 19 to 25° C. under laboratory conditions (fluorescent overhead lighting ˜10-12:14-12 and window light/indirect sunlight). Slugs were fed on carrot pieces. Slugs were then starved for a period of 48 hours before treatment. Compound I-10 was diluted with deionized water. The treatment solution had 0.01% (v/v) adjuvant, Kinetic® (adjuvant comprising 99% by weight of a proprietary blend of polyalkyleneoxide modified polydimethylsiloxane and nonionic surfactants), added to the final volume of a treatment. Potted cabbage plants at 2-4 true leaf stages were treated with a spray chamber application at 300 L/ha using a three nozzle “U” shaped boom which applies the treatment solution on top and to the underside of the leaves. The spray boom is fitted with ConeJet® VisiFlo®: TXVS6 hollow cone spray nozzles. After application, in each case 3 plants were put in 3 gallon plastic buckets, and then five slugs were placed onto the leaves of the treated plants at 0.5 hours after treatment. After infestation, treated plants and slugs were held at 25° C. at 70% relative humidity in a Percival with 14:10 Day:Night light cycle for 6 days after treatment. The slug mortality was recorded at 6 days after treatment, compared to untreated control plants.

The results are shown in Table A below.

TABLE A

| Treatment | Application Rates (g a.i./ha) | % Mortality |
|---|---|---|
| Check | — | 0 |
| Compound I-10 | 300 | 67 |

1. A pesticide comprising
(a) a compound of formula I 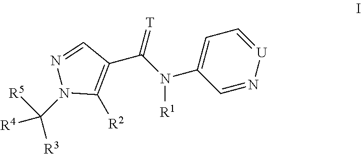
wherein
U is N or CH;
T is O or S;
R¹ is H, C₁-C₂-alkyl, or C₁-C₂-alkoxy-C₁-C₂-alkyl;
R² is CH₃;
R³ is C₁-C₆-alkyl, C₁-C₆-haloalkyl, C₁-C₂-alkoxy-C₁-C₂-alkyl, C₂-C₆-alkenyl, C₂-C₆-alkynyl, C₃-C₆-cycloalkyl, C₃-C₆-cycloalkenyl, C₁-C₆-alkoxy, CN, NO₂, S(O)ₙR^{b}, wherein the C-atoms may be unsubstituted, or partially or fully substituted by R^{a};
wherein
R^{a} is halogen, CN, NO₂, C₁-C₂-alkyl, C₁-C₂-haloalkyl, C₁-C₄-alkoxy, C₁-C₂-haloalkoxy, or S(O)ₙR^{b};
n is 0, 1, or 2; and
R^{b} is hydrogen, C₁-C₂-alkyl, C₁-C₂-haloalkyl, C₃-C₆-cycloalkyl, or C₁-C₄-alkoxy;
R⁴ is C₁-C₄-alkyl, or a group mentioned for R³; or
R⁵ is H, or a group mentioned for R⁴;
or a stereoisomer, salt, tautomer or N-oxide thereof; or
(b) a compound selected from
(b5) 2-(3-pyridyl)-5-[6-(2-pyridyl)-2-pyridyl]thiazole; (b6) N-Ethyl-N-[4-methyl-2-(3-pyridyl)thiazol-5-yl]-3-methylsulfanyl-propanamide; (b7) N-Methyl-N-[4-methyl-2-(3-pyridyl)thiazol-5-yl]-3-methylsulfanyl-propanamide; (b8) N,2-Dimethyl-N-[4-methyl-2-(3-pyridyl)thiazol-5-yl]-3-methylsulfanyl-propanamide; (b9) N-Ethyl-2-methyl-N-[4-methyl-2-(3-pyridyl)thiazol-5-yl]-3-methylsulfanyl-propanamide; (b10) N-[4-Chloro-2-(3-pyridyl)thiazol-5-yl]-N-ethyl-2-methyl-3-methylsulfanyl-propanamide, (b11) N-[4-Chloro-2-(3-pyridyl)thiazol-5-yl]-N,2-dimethyl-3-methylsulfanyl-propanamide; (b12) N-[4-Chloro-2-(3-pyridyl)thiazol-5-yl]-N-methyl-3-methylsulfanyl-propanamide; (b13) N-[4-Chloro-2-(3-pyridyl)thiazol-5-yl]-N-ethyl-3-methylsulfanyl-propanamide; (b14) N-[4-chloro-2-(3-pyridyl)thiazol-5-yl]-N-cyclopropyl-2-methyl-3-methylsulfanyl-propanamide; (b15) N-[3-Chloro-1-(pyridine-3-yl)-1H-pyrazol-4-yl]-N-ethyl-3-((3,3,3-trifluoropropyl)thio)propanamide; (b16) N-[3-Chloro-1-(pyridine-3-yl)-1H-pyrazol-4-yl]-N-ethyl-3-((3,3,3-trifluoropropyl)sulfanyl)propanamide; (b17) N-[3-Chloro-1-(pyridine-3-yl)-1H-pyrazol-4-yl]-3-(((2,2-difluorocyclopropyl)methyl)thio)-N-ethylpropanamide; and (b18) N-[3-Chloro-1-(pyridine-3-yl)-1H-pyrazol-4-yl]-3-(((2,2-difluorocyclopropyl)methyl)sulfinyl)-N-ethylpropanamide;
wherein the pesticide is suitable for protecting plants, the plant propagation material thereof, or soil or water, in which the plants are growing, against the attack or infestation by gastropods.

2. The pesticide according to claim 1, wherein the pesticide is a compound of formula I, wherein U is N, T is O, R² is CH₃ and R⁵ is H, said compounds being compounds of formula IA.

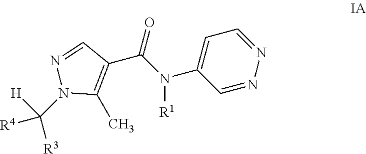

3. The pesticide according to claim 1, wherein the pesticide is a compound of formula I, which is selected from the following formula IA compounds I-1 to I-17 listed below.

| No | R¹ | R³ | R⁴ |
|---|---|---|---|
| I-1 | CH₃ | CH₃ | CH₃ |
| I-2 | CH₃ | CF₃ | CH₃ |
| I-3 | CH₃ | CH(CH₃)₂ | CH₃ |
| I-4 | CH₃ | 1-CN—c-C₃H₄ | CH₃ |
| I-5 | CH₃ | CHFCH₃ | CH₃ |
| I-7 | CH₂CH₃ | CH₃ | CH₃ |
| I-8 | CH₂CH₃ | CF₃ | CH₃ |
| I-9 | CH₂CH₃ | CH(CH₃)₂ | CH₃ |
| I-10 | CH₂CH₃ | 1-CN—c-C₃H₄ | CH₃ |
| I-11 | CH₂CH₃ | CHFCH₃ | CH₃ |
| I-13 | CH₂OCH₃ | CH₃ | CH₃ |
| I-14 | CH₂OCH₃ | CF₃ | CH₃ |
| I-15 | CH₂OCH₃ | CH(CH₃)₂ | CH₃ |
| I-16 | CH₂OCH₃ | 1-CN—c-C₃H₄ | CH₃ |
| I-17 | CH₂OCH₃ | CHFCH₃ | CH₃ |

4. A method for controlling gastropods comprising contacting the gastropods or their food supply, habitat or breeding ground; or plants or plant propagation material thereof, or soil or water, wherein the gastropods are present or may occur, with a molluscicidally effective amount of a pesticide as defined in claim 1.

5. The method according to claim 4, wherein the gastropods are selected from the group consisting of Achatina spp., Agriolimax spp., Arion spp., Biomphalaria spp., Bradybaena spp., Bulinus spp., Cantareus spp., Cepaea spp., Cernuella spp., Cochlicella spp., Cochlodina spp., Deroceras spp., Discus spp., Euomphalia spp., Galba spp., Helicella spp., Helicigona spp., Helicodiscus spp., Helix spp., Limax spp., Lymnaea spp., Milax spp., Oncomelania spp., Opeas spp., Oxyloma spp., Pomacea spp., Succinea spp., Tandonia spp., Theba spp., Vallonia spp., and Zonitoides spp.

6. The method according to claim 4, wherein the plants are selected from the group consisting of cereals including wheat, maize, barley, oat, rye, sorghum, and rice; oil crops including rapeseed and turnip rape, mustard, poppies, olives, sunflowers, coconuts, castor, cacao and peanuts; fiber plants including cotton, flax, hemp or jute; leaf vegetables including lettuce, spinach, and cabbage; bud vegetables including Brussels sprouts; tuber vegetables including potatoes; root vegetables including turnips, beets such as sugar or fodder beet, radishes, carrots, and swedes; leguminous vegetables including lentils, alfalfa, soybeans, beans and peas; stem shoot vegetables including asparagus; marrows including pumpkins, cucumbers and melons; laurels including avocado, cinnamonium and camphor; tobacco, nuts, coffee, egg plants, sugar cane, tea, pepper, grapevines, hops, the banana family, and latex plants; fruits including pome fruit, stone fruit, apples, pears, plums, peaches, almonds, cherries, strawberries, raspberries and blackberries; citrus fruits including oranges, lemons, grapefruits and tangerines; clover and newly sown leys; medicinal herbs; and ornamentals.

7. The method according to claim 4, wherein the pesticide is applied in combination with at least one additional molluscicide.

8. The method according to claim 4, wherein the pesticide additionally controls aphids.

9. The method according to claim 4, wherein the pesticide is applied to plant propagation material.

10. A composition comprising
(i) at least one pesticide as defined in claim 1; and
(ii) at least one mollusc attractant.

11. The composition according to claim 10, wherein the mollusc attractant is
(iia) a phagostimulant, which is selected from the group consisting of ground cereals including cereal flours, cereal brans, and rice starch; crushed soybeans; crushed rapeseed; fish meal; molasses; and any combination thereof; or
(iib) an attractant, which is selected from the group consisting of plant materials including grass and grass seeds; extracts from fruits, cabbage, herbs, tomatoes or potatoes; beer; yeast; extracts from dead slugs; and any combination thereof; or
(iic) a combination of (iia) and (iib).

12. The composition according to claim 10, wherein the mollusc attractant is
(iia) a phagostimulant, which is selected from the group consisting of wheat flour, barley flour, rye flour, rice starch, and combinations thereof.

13. The composition according to claim 20, wherein the at least one additive is selected from bittering agents and repellents.

14. The composition according to claim 10, wherein the composition further comprises at least one additional molluscicide.

15. The method according to claim 4, wherein the pesticide is applied in the form of a composition as defined in claim 10, and wherein said composition is applied to the soil, wherein plants are growing or are intended to grow.

16. The method according to claim 5, wherein the gastropods are selected from the group consisting of Achatina spp., Agriolimax spp., Arion ater, Arion hortensis, Arion lusitanicus, Bradybaena spp., Cantareus spp., Cepaea spp., Cernuella spp., Cochlicella spp., Cochlodina spp., Deroceras agreste, Deroceras reticulatum, Discus spp., Euomphalia spp., Helicella spp., Helicigona spp., Helicodiscus spp., Helix spp., Limax spp., Milax spp., Opeas spp., Oxyloma spp., Tandonia spp., Theba spp., Vallonia spp., and Zonitoides spp.

17. The method according to claim 16, wherein the gastropods are selected from the group consisting of Agriolimax spp., Arion ater, Arion hortensis, Arion lusitanicus, Helix spp., Limax spp., Milax spp., Deroceras agreste, and Deroceras reticulatum.

18. The method according to claim 6, wherein the plant is selected from cereals, rapeseed, leaf vegetables, and ornamentals.

19. The method according to claim 9, wherein the pesticide is applied to the seeds of a plant, or to the foliage of a plant, or to the soil around the base of a plant stem, or around a base of a seedling stem.

20. The composition according to claim 10, further comprising (iii) at least one additive.
